(19) United States
(12) Patent Application Publication
(10) Pub. No.: US 2024/0261340 A1
(43) Pub. Date: Aug. 8, 2024

(54) Cutibacterium Acnes Biomimetic, Prebiotic, Probiotic and Postbiotic Compositions for Treatment of Skin Conditions

(71) Applicant: Johnson & Johnson Consumer Inc., Skillman, NJ (US)

(72) Inventors: Georgios N. Stamatas, Issy-les-Moulineaux (FR); Jose Serrano, Issy-les-Moulineaux (FR)

(21) Appl. No.: 18/429,870

(22) Filed: Feb. 1, 2024

Related U.S. Application Data
(60) Provisional application No. 63/443,595, filed on Feb. 6, 2023.
(60) Provisional application No. 63/443,796, filed on Feb. 7, 2023.

(51) Int. Cl. A61K 35/74; A61K 8/99; A61K 45/06; A61Q 19/00; C12N 1/20; C12R 1/01

(52) U.S. Cl. CPC A61K 35/74; A61K 8/99; A61K 45/06; A61Q 19/00; C12N 1/20; C12R 2001/01

(57) ABSTRACT

Biomimetic, prebiotic, probiotic and postbiotic compositions and methods of use thereof are disclosed. The biomimetic, prebiotic, probiotic and postbiotic compositions may include bacteria of the genera Cutibacterium acnes (C. acnes), its culture and/or components thereof; and/or a substance that induces the growth or activity of C. acnes. The biomimetic, prebiotic, probiotic and postbiotic compositions may be used in preventing, improving and/or treating such conditions such as atopic dermatitis.

### CROSS-REFERENCE TO RELATED APPLICATIONS

This application claims priority under 35 U.S.C. § 119(e) to U.S. Provisional Application No. 63/443,595 filed on Feb. 6, 2023, and U.S. Provisional Application No. 63/443,796 filed on Feb. 7, 2023, each of which are incorporated herein by reference in their entireties.

### FIELD OF THE INVENTION

The present disclosure relates generally to biomimetic, prebiotic, probiotic, and postbiotic compositions and methods of use thereof. More specifically, the present disclosure relates to biomimetic, prebiotic, probiotic, and postbiotic compositions that may include bacteria of the species Cutibacterium acnes (C. acnes), its culture, and/or components thereof. The biomimetic, prebiotic, probiotic, and postbiotic compositions may be used in preventing, improving, and/or treating skin conditions such as atopic dermatitis.

### BACKGROUND

The skin is the human body's largest organ, colonized by a diverse milieu of microorganisms. Colonization is driven by the ecology of the skin surface, which is highly variable depending on topographical location, endogenous host factors and exogenous environmental factors. Microorganisms including bacteria, fungi, and viruses are known to colonize the skin. The skin and the gastrointestinal (“GI”) tract of humans are colonized by a diverse array of microorganisms beginning at the time of birth when an infant is exposed to the maternal microflora and other environmental microbes. From the time of initial colonization, the human microbiome remains in a state of flux where the composition of the resident microflora changes over time in response to factors intrinsic and extrinsic to the host.

Human skin continuously undergoes self-renewal, so resident microbial cells are shed in the process. Most of the microbes found on the skin are harmless to healthy individuals. Some are considered to be mutualistic organisms and confer health benefits to the skin by secreting, for example, antibacterial substances, preventing pathogen colonization, and influencing host immune responses. On the other hand, commensal microorganisms can cause disease and infection if the physical barrier of the skin has been compromised due to trauma or injury.

Atopic dermatitis is one of the most common chronic diseases in the population. Atopic dermatitis is characterized by a set of clinical signs, the most important of which are pruritus and eczematous lesions, which may be acute, subacute or chronic. Atopic dermatitis typically begins at around three months of age, but sometimes in the first few weeks of life, while the physical barrier of the skin undergoes structural and functional maturation processes. Atopic dermatitis progresses in alternating relapse and remission phases. Depending on the child and the severity of the condition, atopic dermatitis may last from several months to several years, while a substantial percentage will persist into adulthood.

Atopic dermatitis is, first and foremost, a chronic inflammatory dermatological disease combining impairment of the skin barrier and skin inflammation. In a first sensitization phase, the skin barrier defect allows allergens to penetrate through the skin. Allergens that penetrate the upper layers of the epidermis are processed (internalized) by epidermal Langerhans cells and dermal dendritic cells. Langerhans cells are antigen-presenting cells that are able to capture skin antigens, prepare them and present them to T lymphocytes. This presentation along with direct activation of epidermal alarmins such as IL-33, IL-25 and TSLP lead to activation of the Th2 response, which results in the production of inflammatory cytokines such as IL-4, IL-5 and IL-13.

In all periods of activity of the disease, bacterial or viral skin superinfections are the most common complications. The skin of atopic dermatitis patients is highly susceptible to secondary infections, which then tend to become more widespread. For example, the bacterium Staphylococcus aureus is a major cause of skin infections. It commonly colonizes the skin of atopic dermatitis patients, whereas it is only transiently present on healthy skin. The bacterium then secretes virulence factors that further reduce the barrier function, exacerbating the disease and contributing to its chronicity. In addition, S. aureus is usually found in atopic dermatitis patients including in a form of homogeneous biofilms, a form resistant to host defenses and treatments.

Several treatment options currently exist with varying degrees of success. For example, “probiotics” are live microorganisms (typically bacteria) that provide health benefits when used, generally by improving or restoring microbiota. “Postbiotics” are soluble factors (metabolic products or byproducts), secreted by bacteria, or released after bacterial lysis that provide physiological benefit to a host. “Prebiotics” are substances that selectively induce the growth or activity of beneficial microorganism. Colloidal oat compositions have been found to increase the growth of C. acnes in an in vitro study. Emollient use has been correlated with an increased richness and a trend toward higher bacterial diversity as compared to no emollient use in infants at risk for developing atopic dermatitis. Some studies have shown that microbial richness is significantly greater with infant wash and lotion than with wash alone. Both cleansing alone and cleansing and emollient regimens are well tolerated; as skin pH remains slightly acidic throughout in each regimen; mild infant wash+lotion routine helps improve microbial richness, which may contribute to overall skin barrier health by providing the right environment for healthy skin microbes to flourish. Early initiation of daily specialized emollient use until 2 months has been shown to reduce the incidence of AD in the first year of life in high-risk infants.

However, there remains a long-felt need for effective treatments to enhance the health of an individual's skin. The present disclosure provides a composition and method for satisfying such need.

### SUMMARY

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Various examples of the present disclosure provide systems and methods as described herein.

The present disclosure includes a method of preventing, improving and/or treating a skin condition in a subject, comprising administering Cutibacterium acnes (C. acnes), its culture and/or components thereof to the subject.

In one example, the present disclosure also includes a composition for use in preventing, improving and/or treating a skin condition in a subject. The composition comprises Cutibacterium acnes (C. acnes), its culture, and/or components thereof, and a pharmaceutically and/or cosmetically acceptable carrier.

In some examples, the composition further includes a material selected from the group consisting of surfactants, chelating agents, emollients, humectants, conditioners, preservatives, opacifiers, fragrances, and combinations of two or more thereof.

In some examples, the composition is in the form of a solution, suspension, emulsion, lotion, cream, serum, gel, stick, spray, ointment, liquid wash, soap bar, shampoo, hair conditioner, paste, foam, powder, mousse, shaving cream, hydrogel, or film-forming product.

The present disclosure also relates to use of Cutibacterium acnes (C. acnes), its culture and/or components thereof in a cosmetic composition, a food composition or a pharmaceutical composition.

In some examples, the composition is a cosmetic composition or pharmaceutical composition that may be used as a skin external preparation and may be directly applied to an affected area. For example, the composition may be prepared in various forms such as ointment, cream, emulsion and the like.

In some examples, the pharmaceutical composition may be absorbed in the body by oral or parenteral administration, and for example, it may be administered in a non-limiting form such as powders, granules, capsules, injections and the like.

Various forms of the cosmetic composition or pharmaceutical composition may be provided without departing from the scope of the present disclosure. Various examples are possible.

In some examples, the cosmetic composition may inhibit or improve atopic dermatitis, other inflammatory skin diseases or dry skin conditions.

Examples of inflammatory skin diseases include atopic dermatitis, acne, inflammatory keratosis, psoriasis, staphylococcal scalded skin syndrome, contact dermatitis, bacterial dermatitis, impetigo, secondary infection of eczema, folliculitis alopecia, ecthyma, mastitis, folliculitis, and the like.

Other technical features may be readily apparent to one skilled in the art from the following figures, descriptions, and claims.

### BRIEF DESCRIPTION OF THE DRAWINGS

FIG. 1 is a graph showing the effect of pure and conditioned media at varying concentrations on Normal Human Epidermal Keratinocytes (NHEK) using the viability test (MTT) 24 hours after treatment;

FIG. 2 is a graph showing the effect of pure and conditioned media at varying concentrations on NHEK using the viability test (MTT) 6 days after treatment;

FIG. 3 is a diagram showing the steps taken to prepare and collect samples for transcriptomic analysis;

FIG. 4A is a graph illustrating gene set enrichment analysis, i.e., genes up-regulated and genes down-regulated, for RCM C. acnes conditioned broth at 5%;

FIG. 4B is a graph illustrating gene set enrichment analysis for RCM C. acnes conditioned media at 10%;

FIG. 4C is a graph illustrating gene set enrichment analysis for RCM C. acnes conditioned broth at 20%;

FIG. 5 is a graph showing the effect of untreated control, adiponectin 10 ug/ml, and C. acnes conditioned media at varying concentrations on NHEK total lipids after 6 days of treatment;

FIG. 6A is a fluorescence microscopy image showing the effect of an untreated control after 6 days;

FIG. 6B is a fluorescence microscopy image showing the effect of adiponectin 10 ug/ml on NHEK total lipids after 6 days of treatment;

FIG. 6C is a fluorescence microscopy image showing the effect of C. acnes conditioned media at 0.1% on NHEK total lipids after 6 days of treatment;

FIG. 6D is a fluorescence microscopy image showing the effect of C. acnes conditioned media at 0.5% on NHEK total lipids after 6 days of treatment;

FIG. 6E is a fluorescence microscopy image showing the effect of C. acnes conditioned media at 1.0% on NHEK total lipids after 6 days of treatment;

FIG. 7 is a graph showing the effect of untreated control, broth (unconditioned C. acnes culture media) at varying concentrations, C. acnes conditioned media at varying concentrations, and adiponectin 10 ug/ml on NHEK total lipids after 1 day of treatment;

FIG. 8A is a fluorescence microscopy image showing the effect of an untreated control after one day;

FIG. 8B is a fluorescence microscopy image showing the effect of broth 5% on NHEK total lipids after 1 day of treatment;

FIG. 8C is a fluorescence microscopy image showing the effect of broth 10% on NHEK total lipids after 1 day of treatment;

FIG. 8D is a fluorescence microscopy image showing the effect of C. acnes conditioned media at 5% on NHEK total lipids after 1 day of treatment;

FIG. 8E is a fluorescence microscopy image showing the effect of C. acnes conditioned media at 10% on NHEK total lipids after 1 day of treatment;

FIG. 8F is a fluorescence microscopy image showing the effect of adiponectin 10 ug/ml on NHEK total lipids after 1 day of treatment;

FIG. 9 shows Principle Component Analysis (PCA) scores of lipidomic analysis of NHEK samples collected following various tested conditions;

FIG. 10 shows Principle Component Analysis (PCA) scores of lipidomic analysis of NHEK samples collected at different timepoints following exposure to various tested conditions; and

FIG. 11 is a table showing lipid classes from the lipidomic analysis of NHEK samples that showed statistically significant difference between the conditions of exposure to C. acnes conditioned media and untreated control (for samples collected at day 2 and day 3).

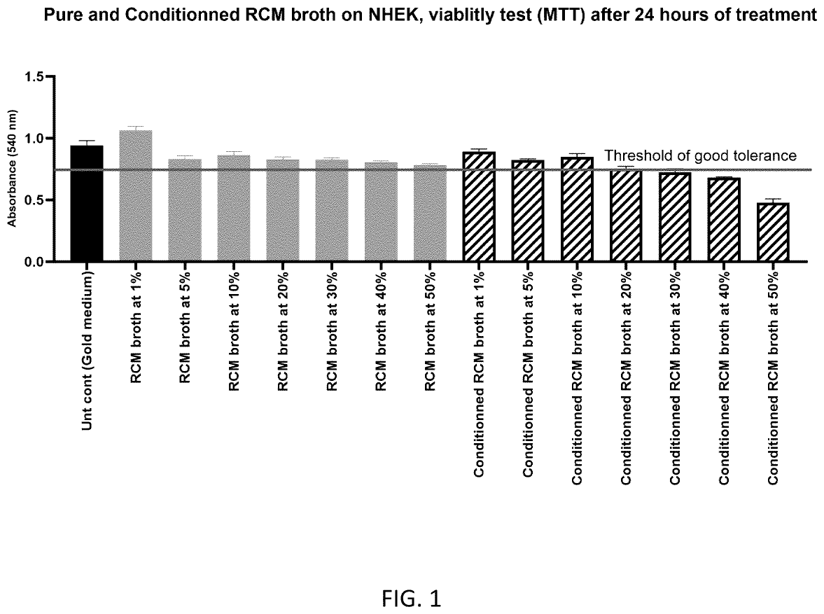

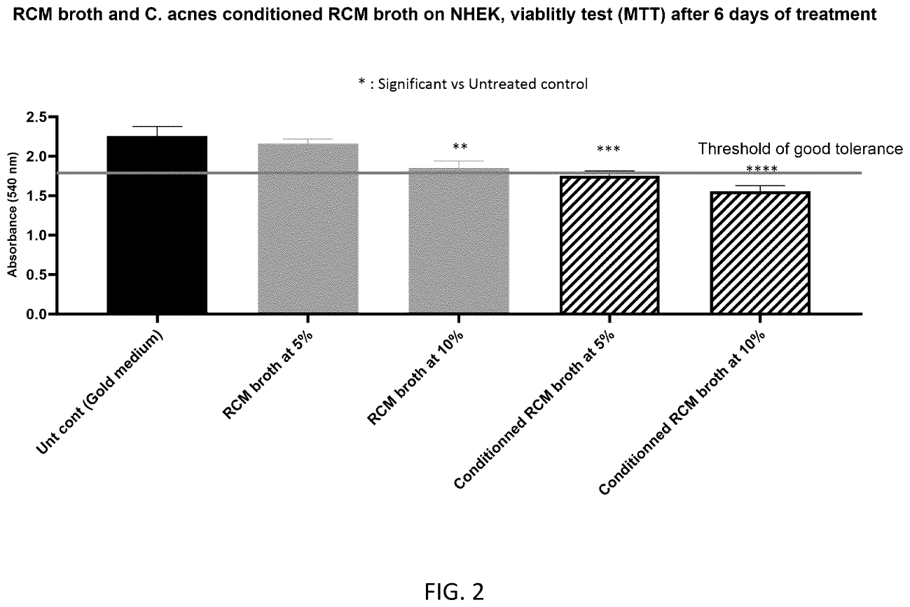

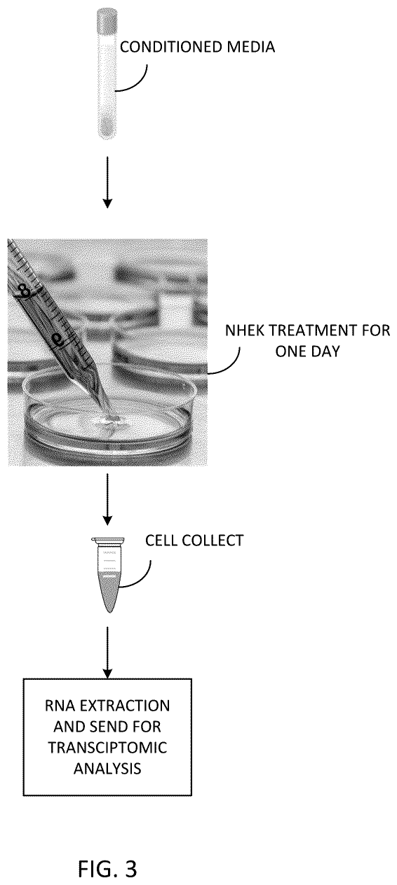

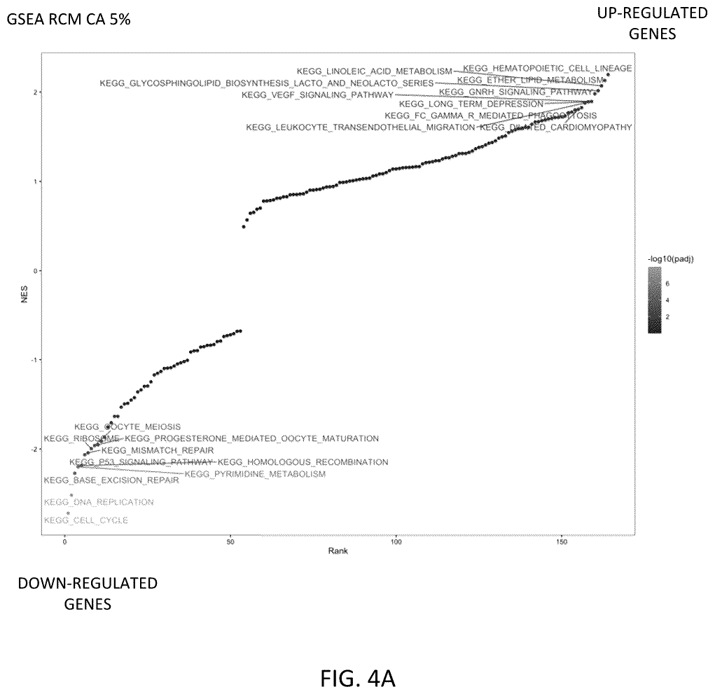

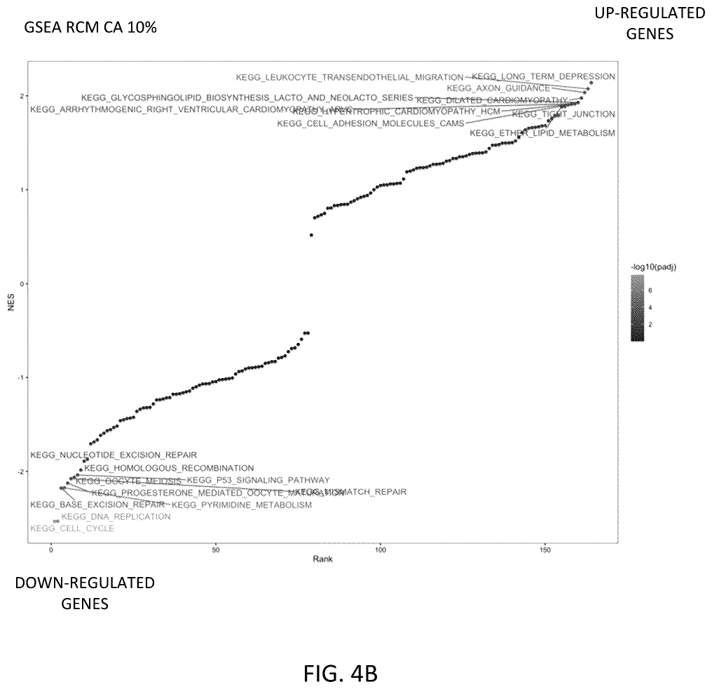

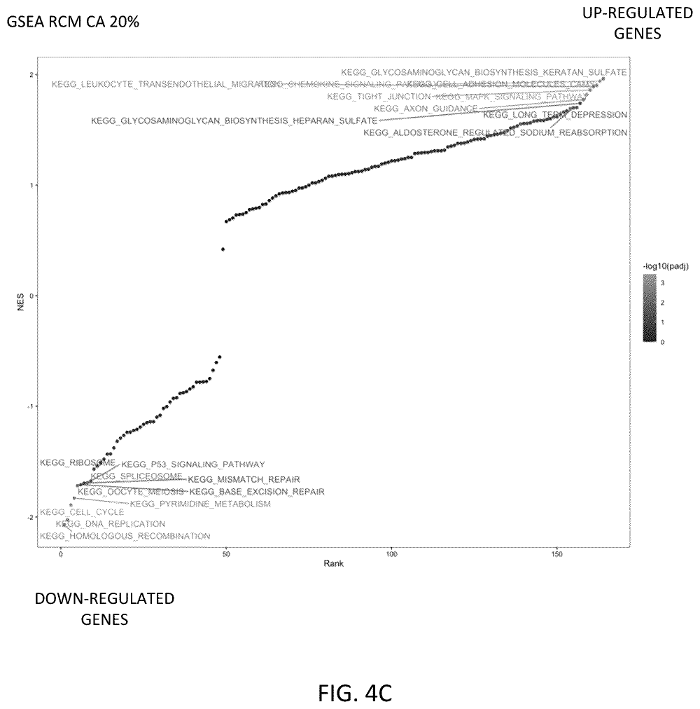

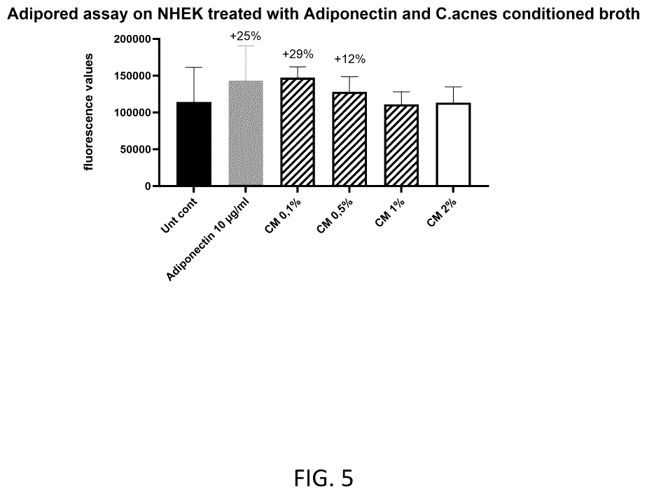

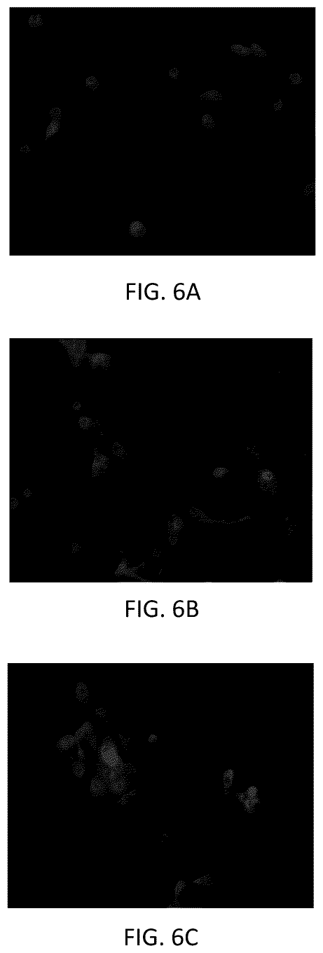

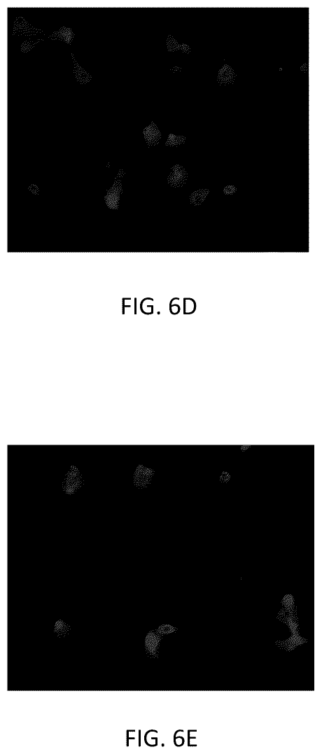

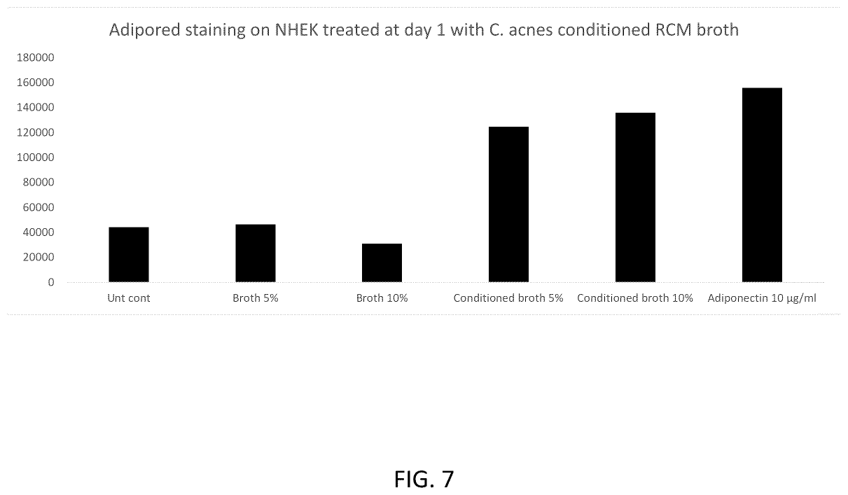

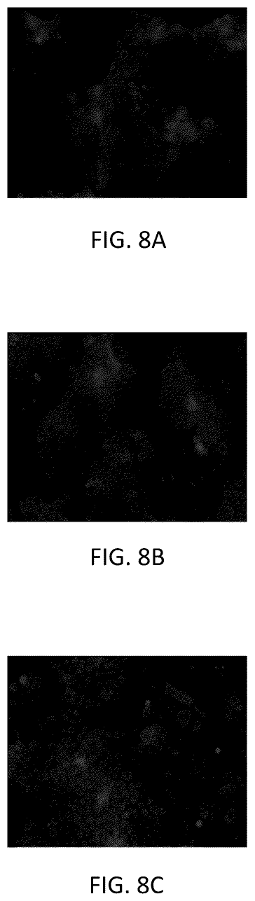

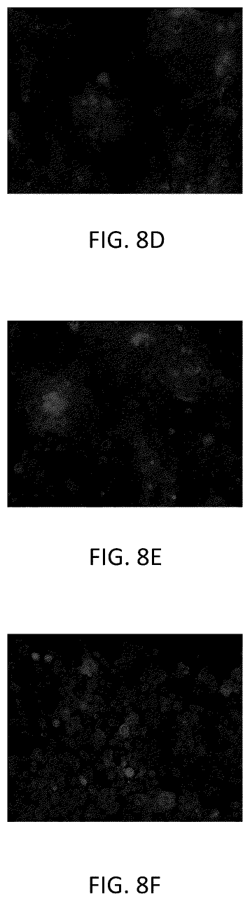

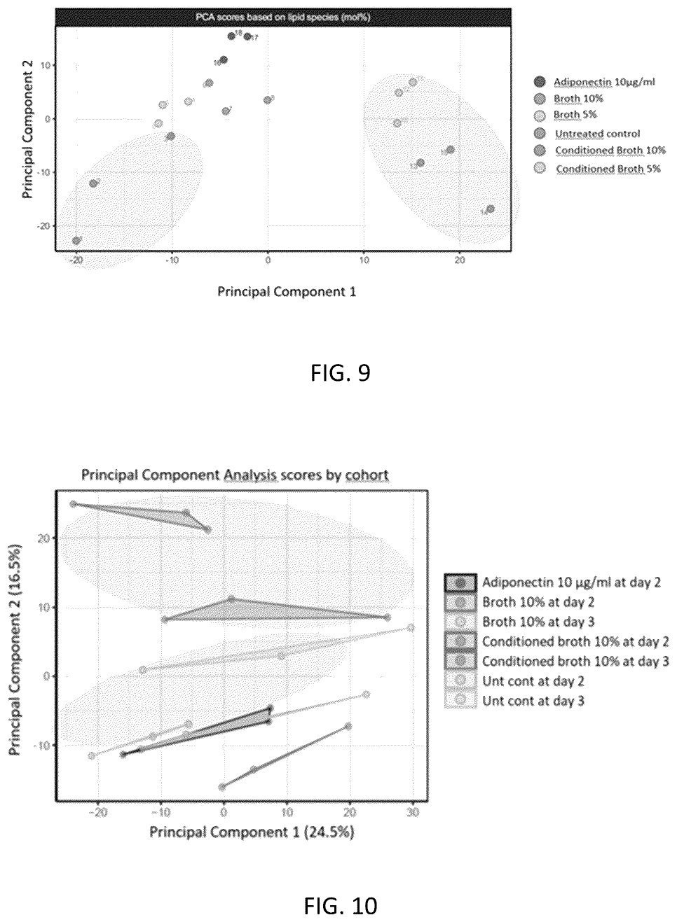

### DETAILED DESCRIPTION

Hereinafter, the present disclosure will be described by the following examples and the like in order to described it more specifically. However, the examples according to the present disclosure may be modified to various other forms, and the scope of the present disclosure should not be construed as being limited to the examples described below. The examples of the present disclosure are illustratively provided in order to facilitate a specific understanding of the present disclosure.

The various implementations and examples will be described in detail with reference to the accompanying drawings. Wherever possible, the same reference numbers will be used throughout the drawings to refer to the same or like parts. References made throughout this disclosure relating to specific examples and implementations are provided solely for illustrative purposes but, unless indicated to the contrary, are not meant to limit all examples.

As used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein. Unless otherwise indicated, the term “at least” preceding a series of elements is to be understood to refer to every element in the series. Those skilled in the art will recognize, or be able to ascertain using no more than routine experimentation, many equivalents to the specific examples of the disclosure described herein. Such equivalents are intended to be encompassed by the present disclosure.

The term “about” or “approximately” as used herein means within 20%, preferably within 15%, more preferably within 10%, preferably within 5%, and preferably within 1% of a given value or range.

The term “and/or” wherever used herein includes the meaning of “and”, “or” and “all or any other combination of the elements connected by said term”. “Biomimetics” are substances that (1) are synthesized by artificial means; and (2) mimic natural substances having known biological function. Ahmadi et al., Supramolecular architecture of a multi-component biomimetic lipid barrier formulation, Journal of Colloid and Interface Science, Volume 587, April 2021, Pages 597-612, discloses multi-component lipid/surfactant formulations that may be employed in ‘biomimetic’ skin care preparations. Di Lorenzo et al., Biomimetic endorphin for wrinkle lifting in a daily cosmetic remedy, Dermatol Res Skin Care. 2022;6(2): 106, discloses biomimetic peptides for cosmetic use. U.S. Pat. No. 6,544,401 to Henceforth Hibernia, Inc. discloses biomimetic water solutions and their use in health and beauty products.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

“Conditioned medium” is media containing biologically active components, including metabolites, obtained from previously cultured cells or tissues that have released into the media substances that affect certain cell functions (e.g., growth, lysis).

“Culture” includes (1) a total medium comprising a strain, strain extract, its metabolite, extra nutriments and the like, obtained by culturing the strain during a certain period in a medium capable of supplying nutrients so that the strain can grow and survive and (2) a solution in which the strain is eliminated after culturing the strain. The culture for the composition of the present disclosure may use a medium easily selected according to a purpose by those skilled in the art among media used for microorganism culturing, and preferably, it may use a medium used for Cutibacterium culturing, and more preferably, it may use RCM (Reinforced Clostridium Medium) medium, TSB (Tryptic soy broth) or BHI (Brain Heart Infusion) medium, but is not limited thereto.

“Diffusible mediator” is an agent that is either locally released or carried in blood or tissue fluids that may participate in initiating, perpetuating, or aggravating a biological process.

“Gene Set Enrichment Analysis” (GSEA) is a computational method that determines whether an a priori defined set of genes shows statistically significant, concordant differences between two biological states.

“MTT assay” is a colorimetric assay for assessing cell metabolic activity. NAD(P)H-dependent cellular oxidoreductase enzymes may under defined conditions, reflect the number of viable cells present. These enzymes are capable of reducing the tetrazolium dye MTT, which is chemically 3-(4,5-dimethylthiazol-2-yl)-2,5-diphenyltetrazolium bromide, to its insoluble formazan, which has a purple color. It has also been found of MTT reduction to formazan in lipidic cellular structures without apparent involvement of oxidoreductases.

“Principal component analysis” (PCA) is a technique for reducing the dimensionality of large datasets to increase interpretability.

### EXAMPLE

In a clinical study, the present inventors found that Cutibacterium acnes (C. acnes) presence correlates positively with an enriched lipid signaling pathway (PF Roux, T Oddos, and G Stamatas, “Deciphering the Role of Skin Surface Microbiome in Skin Health: An Integrative Multiomics Approach Reveals Three Distinct Metabolite-Microbe Clusters”, J Invest Dermatol, 142(2), 469-479, 2021). To test the existence of a causal relationship that could explain this observation, it was hypothesized that C. acnes stimulated lipid production in keratinocytes through a diffusible mediator. To test this hypothesis, three complementary types of experiments involving human keratinocytes treated with C. acnes conditioned media at non-cytotoxic concentrations were conducted. A transcriptomic analysis of the treated keratinocytes was conducted to test if genes related to lipid metabolism were up-regulated, fluorescence assays using a lipid stain were conducted to observe if there was intracellular lipid accumulation, and Lipidomic analysis of the treated keratinocytes was conducted to test if there was dose and time dependent effect.

A cytotoxicity test was first performed. C. acnes (ATCC 6919) was cultivated in RCM broth, Biomerieux (Craponne, France) at 2.03×10⁹ CFU. After centrifugation and filtration of the RCM broth to eliminate the live bacteria, the “conditioned media =conditioned broth” that was frozen at −80° C. was isolated for further testing on Normal Human Epidermal Keratinocytes (NHEK). Viability tests were performed on NHEK to determine non-cytotoxic concentrations.

A transcriptomic analysis was then performed. For example, MTT tests were performed to estimate non-cytotoxic concentrations of RCM broth and C. acnes conditioned broth. The RCM broth alone is well tolerated whatever the test concentration, while the correct concentration of C. acnes conditioned media depends on the duration of the treatment. The cytotoxic effect observed with conditioned broth demonstrates that C. acnes releases virulence factors.

Differentiated NHEK were treated for 1 day with RCM broth (non-conditioned media) and C. acnes conditioned broth (conditioned media) at concentrations of 0, 5, 10, and 20%. The experiment was performed using one replicate/condition. NHEK were harvested after PBS rinsing, using 0.05% Trypsin treatment at 37° C. to detach cells and addition of 10% calf serum in DMEM for trypsin neutralization (Lonza, Verviers Belgium for all the reagents). Cells were centrifuged, the supernatants were eliminated and replaced by RLT buffer (Qiagen, Courtaboeuf France) supplemented with 1% β mercapto-ethanol (Sigma-Aldrich, St Quentin Fallavier, France). RNA was extracted using Qiacube HT extractor and its specific reagents and protocol (Qiagen, Courtaboeuf, France). RNA aliquots were sent to Genex (Longjumeau, France) for transcriptomic analysis. It was observed that, independent of the concentration of the conditioned media, genes involved in DNA replication were down-regulated, i.e., cells reduced their turnover, and genes involved in lipid production were up-regulated.

Lipid accumulation using a fluorescence assay was then assessed. In particular, following the results of the transcriptomic analysis, NHEK were treated for 6 days with conditioned media at concentrations of 0.1, 0.5, 1, and 2%. Adiponectin 10 μg/ml (Sigma, St Quentin Fallavier, France), known in the literature to induce lipid production in cell cultures, was used as positive control. To quantify and visualize total lipids after the 6 days of treatment, cells were treated with AdipoRed™ Assay Reagent (Lonza, Verviers, Belgium) a lipophilic reagent which fluoresces (excitation: 485 nm, emission: 572 nm). The intensity of the fluorescence is proportional to the lipid concentration and can be measured using a spectrophotometer (Envision, Perlin Elmer, Villebon-sur-Yvette, France). Images were acquired using a fluorescence microscope (Axio imager, ZEISS, Rueil Malmaison France) with magnification ×20.

Adiponectin induced an increase in fluorescence signal after 6 days of treatment (+25%) which validated the test. Conditioned media tested at 0.1% (non-toxic concentration) also showed an increased fluorescence signal compared to untreated control (+29%). Fluorescence microscopy images also showed that treatment with conditioned media at 0.1% and 0.5% increased the intracellular fluorescence at day 6 compared to untreated control. This experiment was repeated increasing the concentration of the conditioned media to 5% and 10% and reducing the time of treatment to 1 day. Non-conditioned media at 5% and 10% were also tested as negative controls and Adiponectin (10 μg/ml) as positive control.

Fluorescence intensity values measured by the spectrophotometer increased dramatically for the conditioned media at 5% and 10% by respectively +181% and +206% versus the untreated control. Non-conditioned media did not induce any increase in fluorescence. Adiponectin induced a 250% increase in fluorescence which validated the test. Fluorescence microscopy images of these test conditions at day 1 confirmed an increase in the fluorescence induced by the conditioned media.

Lipidomic analysis was employed to further our understanding of the classes of lipids produced by the keratinocytes treated with C. acnes conditioned media, as well as investigate whether a dose dependent and time dependent effect existed. NHEK were treated for 1 day with RCM (non-conditioned) broth and C. acnes conditioned broth at 0, 5, and 10% (3 replicates/condition). Adiponectin 10 μg/ml was used as positive reference. Keratinocytes were dissociated using PBS (rinsing), Trypsin (cell detachment) and addition of DMEM supplemented with 10% calf serum (trypsin neutralisation). After centrifugation, supernatants were eliminated, and PBS was added to obtain a suspension at 3000 cells/μl.

300 μl of these suspensions were sent to Lipotype GmbH (Dresden, Germany) for lipid analysis. Principle Component Analysis (PCA) scores based on lipid species showed clustering of the tested conditions and that cells treated with broth alone shift their lipid profile compared to untreated control. Cells treated with conditioned broth have a markedly different lipid profile compared to untreated or non-conditioned broth treated cells. The distance from the untreated cluster is greater at higher concentrations, indicating a dose-dependent effect.

In another experiment, NHEK were treated with RCM (non-conditioned) broth and C. acnes conditioned broth at 0 and 10% (3 replicates/condition). Adiponectin 10 μg/ml was used as positive reference. Cells were harvested at day 2 and 3 using the same protocol as previously described. Following the same proceedure of lipidomic analysis, at the PCA plot, it can be seen that there is a shift in the lipid profile away from the untreated control in a time-dependent fashion.

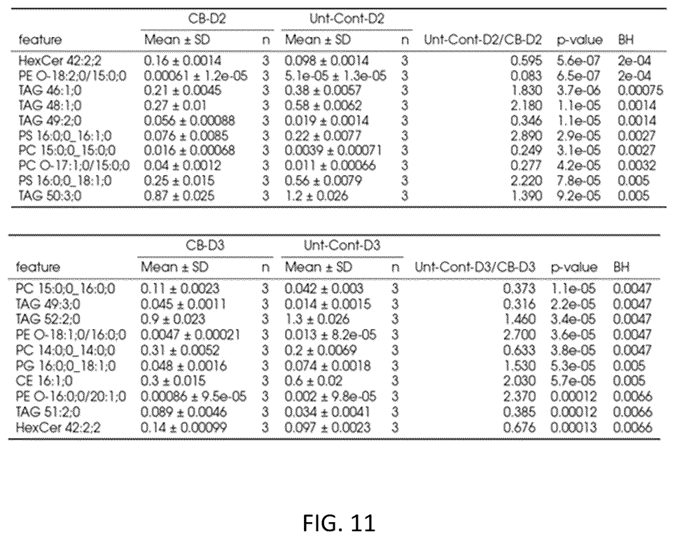

The lipid classes that were induced in keratinocytes were further examined by the conditioned media treatment. Results are summerized in the tables illustrated in FIG. 11, showing the ten most significant lipid species. In particular, a comparison was done between i) the conditioned media at day 2 versus the untreated control at day 2, and ii) the conditioned media at day 3 versus the untreated control at day 3.

As shown herein, it is concluded that treating normal human epidermal keratinocytes with C. acnes conditioned media resulted in interesting and unexpected results in relation to the untreated control. These results include the upregulation of genes involved in lipid metabolism, an increased fluorescence values indicating enhanced lipid production, confirmation of the accumulation of intracellular lipids by fluorescence microscopy images, a dose dependent effect in lipid composition analysis, and a time dependent effect in lipid composition analysis. Accordingly, these results demonstrate that C. acnes stimulates lipid production in human keratinocytes through diffusible mediators.

Any suitable carrier may be used in the composition or compositions described herein. In some examples, the carrier is a cosmetically-acceptable carrier. As will be recognized by those of skill in the art, cosmetically acceptable carriers comprise carriers that are suitable for use in contact with the body, in particular the skin, without undue toxicity, incompatibility, instability, irritation, allergic response, and the like. A safe and effective amount of carrier is from about 50, 55, 60, 65, 70, 75, 80, 85, 90, 95, or 98% to about 85, 90, 95, 98, 99, 99.1, 99.5 or 99.9% by weight of the composition.

The carrier can be in a wide variety of forms. For example, carriers in the form of emulsions, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps using a Brookfield RVT viscometer.

Examples of suitable cosmetically-acceptable carriers include cosmetically acceptable solvents and materials for cosmetic solutions, suspensions, lotions, creams, serums, essences, gels, toners, sticks, sprays, ointments, liquid washes and soap bars, shampoos, hair conditioners, pastes, foams, mousses, powders, shaving creams, wipes, patches, strips, powered patches, micro-needle patches, bandages, hydrogels, film-forming products, facial and skin masks, make-up, liquid drops, and the like. These product types may contain several types of cosmetically-acceptable carriers including, but not limited to solutions, suspensions, emulsions such as micro-emulsions and nano-emulsions, gels, solids, liposomes, other encapsulation technologies and the like.

The following are non-limiting examples of carriers. Other carriers can be formulated by those of ordinary skill in the art. In one example, the carrier contains water. In a further example, the carrier may also contain one or more aqueous or organic solvents. Examples of organic solvents include, but are not limited to: dimethyl isosorbide; isopropyl myristate; surfactants of cationic, anionic and nonionic nature; vegetable oils; mineral oils; waxes; gums; synthetic and natural gelling agents; alkanols; glycols; and polyols. Examples of glycols include, but are not limited to, glycerin, propylene glycol, butylene glycol, pentalene glycol, hexylene glycol, polyethylene glycol, polypropylene glycol, diethylene glycol, triethylene glycol, capryl glycol, glycerol, butanediol and hexanetriol, and copolymers or mixtures thereof. Examples of alkanols include, but are not limited to, those having from about 2 carbon atoms to about 12 carbon atoms (e.g., from about 2 carbon atoms to about 4 carbon atoms), such as isopropanol and ethanol. Examples of polyols include, but are not limited to, those having from about 2 carbon atoms to about 15 carbon atoms (e.g., from about 2 carbon atoms to about 10 carbon atoms) such as propylene glycol. The organic solvents may be present in the carrier in an amount, based upon the total weight of the carrier, of from about 1 percent to about 99.99 percent (e.g., from about 20 percent to about 50 percent). Water may be present in the carrier (prior to use) in an amount, based upon the total weight of the carrier, of from about 5 percent to about 95 percent (e.g., from about 50 percent to about 90 percent). Solutions may contain any suitable amounts of solvent, including from about 40 to about 99.99%. Some solutions contain from about 50 to about 99.9%, from about 60 to about 99%, from about 70 to about 99%, from about 80 to about 99%, or from about 90 to 99% of solvent.

A lotion can be made from such a solution. Lotions typically contain at least one emollient in addition to a solvent. Lotions may comprise from about 1% to about 20% (e.g., from about 5% to about 10%) of an emollient(s) and from about 50% to about 90% (e.g., from about 60% to about 80%) of water.

Another type of product that may be formulated from a solution is a cream. A cream typically contains from about 5% to about 50% (e.g., from about 10% to about 20%) of an emollient(s) and from about 45% to about 85% (e.g., from about 50% to about 75%) of water.

Yet another type of product that may be formulated from a solution is an ointment. An ointment may contain a simple base of animal, vegetable, or synthetic oils or semi-solid 10 hydrocarbons. An ointment may contain from about 2% to about 10% of an emollient(s) plus from about 0.1% to about 2% of a thickening agent(s).

The compositions useful in the present disclosure can also be formulated as emulsions. If the carrier is an emulsion, from about 1% to about 10% (e.g., from about 2% to about 5%) of the carrier contains an emulsifier(s). Emulsifiers may be nonionic, anionic or cationic.

Lotions and creams can be formulated as emulsions. Typically such lotions contain from 0.5% to about 5% of an emulsifier(s), while such creams would typically contain from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams, of the oil-in-water type, and water-in-oil type are well-known in the art and are useful in the subject disclosure. Multiphase emulsion compositions, such as the water-in-oil-in-water type or the oil-in-water-in-oil type, are also useful in the subject disclosure. In general, such single or multiphase emulsions contain water, emollients, and emulsifiers as essential ingredients.

The compositions of this disclosure can also be formulated as a gel (e.g., an aqueous, alcohol, alcohol/water, or oil gel using a suitable gelling agent(s)). Suitable gelling agents for aqueous and/or alcoholic gels include, but are not limited to, natural gums, acrylic acid and acrylate polymers, and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically contains between about 0.1% and 5%, by weight, of such gelling agents.

The compositions of the present disclosure can also be formulated into a solid formulation (e.g., a wax-based stick, soap bar composition, powder, or wipe). The composition of the present disclosure can also be combined with a solid, semi-solid, or dissolvable substrate (e.g., a wipe, mask, pad, glove, or strip).

### Other Additives

The compositions of the present disclosure may further comprise any of a variety of additional cosmetically active agents. Examples of suitable additional active agents include: skin lightening agents, darkening agents, additional anti-aging agents, tropoelastin promoters, collagen promoters, anti-acne agents, shine control agents, anti-microbial agents such as anti-yeast agents, anti-fungal, and anti-bacterial agents, anti-inflammatory agents, anti-parasite agents, external analgesics, sunscreens, photoprotectors, antioxidants, keratolytic agents, detergents/surfactants, moisturizers, nutrients, vitamins, energy enhancers, anti-perspiration agents, astringents, deodorants, hair removers, hair growth enhancing agents, hair growth delaying agents, firming agents, hydration boosters, efficacy boosters, anti-callous agents, agents for skin conditioning, anti-cellulite agents, odor-control agents such as odor masking or pH changing agents, and the like.

Examples of various suitable additional cosmetically acceptable actives include hydroxy acids; benzoyl peroxide; D-panthenol; UV filters such as but not limited to avobenzone (Parsol 1789), bisdisulizole disodium (Neo Heliopan AP), diethylamino hydroxybenzoyl hexyl benzoate (Uvinul A Plus), ecamsule (Mexoryl SX), methyl anthranilate, 4-aminobenzoic acid (PABA), cinoxate, ethylhexyl triazone (Uvinul T 150), homosalate, 4-methylbenzylidene camphor (Parsol 5000), octyl methoxycinnamate (Octinoxate), octyl salicylate (Octisalate), padimate O (Escalol 507), phenylbenzimidazole sulfonic acid (Ensulizole), polysilicone-15 (Parsol SLX), trolamine salicylate, Bemotrizinol (Tinosorb S), benzophenones 1-12, dioxybenzone, drometrizole trisiloxane (Mexoryl XL), iscotrizinol (Uvasorb HEB), octocrylene, oxybenzone (Eusolex 4360), sulisobenzone, bisoctrizole (Tinosorb M), titanium dioxide, zinc oxide; carotenoids; free radical scavengers; spin traps; retinoids and retinoid precursors such as retinol, retinoic acid and retinyl palmitate; ceramides; polyunsaturated fatty acids; essential fatty acids; enzymes; enzyme inhibitors; minerals; hormones such as estrogens; steroids such as hydrocortisone; 2-dimethylaminoethanol; copper salts such as copper chloride; peptides containing copper such as Cu: Gly-His-Lys, coenzyme Q10; amino acids such a proline; vitamins; lactobionic acid; acetyl-coenzyme A; niacin; riboflavin; thiamin; ribose; electron transporters such as NADH and FADH2; and other botanical extracts such as oat, aloe vera, Feverfew, Soy, Shiitake mushroom extracts, and derivatives and mixtures thereof.

If present, any additional cosmetically active agent may be present in a composition in any suitable amount, for example, in an amount of from about 0.0001% to about 20% by weight of the composition, e.g., about 0.001% to about 10% such as about 0.01% to about 5%. In some examples, in an amount of 0.1% to 5% and in other examples from 1% to 2%.

Compositions of the present disclosure may include a cosmetically effective amount of one or more additional anti-inflammatory compounds. Examples of suitable anti-inflammatory agents include substituted resorcinols, (E)-3-(4-methylphenylsulfonyl)-2-propenenitrile (such as “Bay 11-7082,” commercially available from Sigma-Aldrich of St. Louis, Missouri), tetrahydrocurcuminoids (such as Tetrahydrocurcuminoid CG, available from Sabinsa Corporation of Piscataway, NJ), extracts and materials derived from the following: Phellodendron amurense Cortex Extract (PCE), Non-Denatured Soy (Glycine max), Feverfew (Tanacetum parthenium), Ginger (Zingiber officinale), Ginkgo (Ginkgo biloba), Madecassoside (Centella asiatica extract ingredient), Cotinus (Cotinus coggygria), Butterbur Extract (Petasites hybridus), Goji Berry (Lycium barbarum), Milk Thistle Extract (Silybum marianum), Honeysuckle (Lonicera japonica), Basalm of Peru (Myroxylon pereirae), Sage (Salvia officinalis), Cranberry Extract (Vaccinium oxycoccos), Amaranth Oil (Amaranthus cruentus), Pomegranate (Punica granatum), Yerbe Mate (Ilex paraguariensis Leaf Extract), White Lily Flower Extract (Lilium candidum), Olive Leaf Extract (Olea europaea), Phloretin (apple extract), Oat Flour (Aveena sativa), Lifenol (Hops: Humulus lupulus) Extract, Bugrane P (Ononis spinosa), Licochalcone (Licorice: Glycyrrhiza inflate extract ingredient), Symrelief (Bisabolol and Ginger extract), combinations of two or more thereof, and the like.

In one example, the anti-inflammatory agent is a resorcinol. Particularly suitable substituted resorcinols include 4-hexyl resorcinol and 4-octylresorcinol, particularly 4-hexyl resorcinol. 4-Hexyl resorcinol is commercially available as “SYNOVEA HR” from Sytheon of Lincoln Park, NJ. 4-Octylresorcinol is commercially available from City Chemical LLC of West Haven, Connecticut.

By “extracts of feverfew,” it is meant extracts of the plant “Tanacetum parthenium,” such as may be produced according to the details set for the in U.S. Pat. No. 7,537,791, entitled “PARTHENOLIDE FREE BIOACTIVE INGREDIENTS FROM FEVERFEW (TANACETUM PARTHENIUM) AND PROCESSES FOR THEIR PRODUCTION.” One particularly suitable feverfew extract is commercially available as about 20% active feverfew, from Integrated Botanical Technologies of Ossining, NY.

A variety of other materials may also be present in the compositions of the present disclosure. In one or more examples, the composition comprises one or more topical ingredients selected from the group consisting of: surfactants, chelating agents, emollients, humectants, conditioners, preservatives, opacifiers, fragrances and the like.

What is claimed is:

1. A method comprising at least one of preventing, improving, or treating a skin condition in a subject, the method comprising:
administering a composition comprising at least one of Cutibacterium acnes (C. acnes), a culture of C. acnes, or components thereof to the subject.

2. The method of claim 1, wherein the composition is a pharmaceutical composition.

3. The method of claim 1, wherein the composition is a cosmetic composition.

4. The method of claim 1, wherein the skin condition is atopic dermatitis.

5. The method of claim 1, wherein the composition is a postbiotic composition derived from a liquid fermentation medium of the C. acnes microorganism.

6. The method of claim 5, wherein the postbiotic composition is prepared by collecting supernatant from the centrifugation of the C. acnes microorganism.

7. The method of claim 1, wherein the composition further comprises at least one of a biomimetic to C. acnes, a culture of C. acnes, or components thereof.

8. The method of claim 7, wherein the biomimetic produces diffusible mediators responsible for induction of lipid production by keratinocytes.

9. The method of claim 1, wherein:
the composition further comprises a prebiotic for use in at least one of preventing, improving, or treating the skin condition in the subject, and
the prebiotic induces at least one of the growth or activity of C. acnes.

10. The method of claim 9, wherein the prebiotic enhances an effect of C. acnes on lipid production by keratinocytes.

11. A composition for use in at least one of preventing, improving, or treating a skin condition in a subject, the composition comprising:
at least one of Cutibacterium acnes (C. acnes), a culture of C. acnes, or components thereof; and
a pharmaceutically and/or cosmetically acceptable carrier.

12. The composition of claim 1, further comprising a material selected from the group consisting of surfactants, chelating agents, emollients, humectants, conditioners, preservatives, opacifiers, fragrances, and combinations of two or more thereof.

13. The composition of claim 1, wherein the composition is in the form of a solution, suspension, emulsion, lotion, cream, serum, gel, stick, spray, ointment, liquid wash, soap bar, shampoo, hair conditioner, paste, foam, powder, mousse, shaving cream, hydrogel, or film-forming product.
